(19) United States
(12) Patent Application Publication
(10) Pub. No.: US 2006/0194856 A1
(43) Pub. Date: Aug. 31, 2006

(54) Use of atii antagonist for the treatment or prevention of metabolic syndrome

(75) Inventor: Anders Ljunggren, MOLNDAL (SE)

Correspondence Address: FISH & NEAVE IP GROUP;ROPES & GRAY LLP, ONE INTERNATIONAL PLACE, BOSTON, MA, 02110-2624, US

(73) Assignee: ASTRAZENECA AB, SODERTALJE (SE)

(21) Appl. No.: 10/550,760

(22) PCT Filed: Mar. 31, 2004
(86) PCT No.: PCT/SE04/00505

§ 371 Date: Sep. 27, 2005

(30) Foreign Application Priority Data
Apr. 3, 2003 (SE) 0300988-3

(51) Int. Cl. A61K 31/4178

(52) U.S. Cl. 514/381

(57) ABSTRACT

The present invention relates to the use of an angiotensin II type 1 receptor antagonist alone, or in combination with a metabolically neutral antihypertensive substance, for the prevention and/or treatment of the metabolic syndrome.

### FIELD OF THE INVENTION

The present invention relates to the use of an angiotensin II type 1 receptor antagonist alone, or in combination with a metabolically neutral antihypertensive substance, for the prevention and/or treatment of the metabolic syndrome.

### BACKGROUND OF THE INVENTION

The metabolic syndrome [JAMA 2001; 285:2486-97] is characterised by high levels of blood fats, high blood pressure, insulin resistance, and central obesity (excessive fat tissue in the abdominal region). Subjects suffering from the metabolic syndrome are also at an increased risk of coronary artery disease and other arteriosclerotic conditions as well as diabetes. It has been proposed that the metabolic syndrome may be genetically based. However, the underlying cause of the disorder is not yet fully understood.

The combination of an angiotensin II type 1 receptor antagonist with a calcium antagonist is known from WO00/02543 A2. The combination has been proposed for use in the treatment of inter alia hypertension, congestive heart failure and myocardial infarction.

The use of a combination of a certain renin inhibitor and at least one therapeutic agent selected from inter alia an AT1-receptor antagonist and an angiotensin converting enzyme inhibitor for the treatment of inter alia diabetic retinopathy, syndrome X and isolated systolic hypertension has been proposed in WO02/40007 A1.

### OUTLINE OF THE INVENTION

The present invention relates to the use of an angiotensin II type 1 receptor antagonist alone, or in combination with a metabolically neutral antihypertensive substance, for the prevention and/or treatment of metabolic syndrome.

The Metabolic Syndrome

The metabolic syndrome is herein defined in accordance with the definition of the World Health Organization, i.e. according to the following criteria [World Health Organization (WHO). Department of Noncommunicable Disease Surveillance. Geneva: WHO 1999 pp 1-59]:
1. Fasting plasma glucose above 6.1 mmol/L
2. Blood pressure above 140/90 mm Hg
3. One or more of the following:
   a) plasma triglycerides above 1.7 mmol/L and/or HDL below 0.9 mmol/L (men), below 1.0 mmol/L (women)
   b) Body mass index above 30 kg/m²
      Fasting Plasma Glucose Level

The fasting plasma glucose level is defined as the concentration of glucose in the plasma of a subject after an overnight's fast. Patients were instructed not to eat breakfast on the morning of the follow-up visits.

Blood Pressure

Blood pressure is defined as the pressure of the blood on the walls of the arteries and is dependent on the energy of the heart action, the elasticity of the walls of the arteries, and the volume and viscosity of the blood. The maximum pressure occurs near the end of the stroke output of the left ventricle of the heart and is termed maximum or systolic pressure. The minimum pressure occurs late in ventricular diastole and is termed minimum or diastolic pressure.

Blood Fats—Plasma Triglycerides

Cholesterol and triglycerides are transported in the body fluids in the form of lipoprotein particles. Lipoproteins are classified according to density: chylomicrons, chylomicron remnants, very low density lipoproteins (VLDL), intermediate-density lipoproteins (IDL), low-density lipoproteins (LDL) and high-density lipoproteins (HDL).

Obesity

Obesity is defined herein as a body mass index (BM) above 30 kg/m². The body mass index is calculated as (body weight in kilograms)/(length in meters)².

Angiotensin II Type 1 Receptor Antagonists

Angiotensin II type 1 receptor antagonists are compounds which are known to interfere with the renin-angiotensin system (RAS) and are used to treat common cardiovascular diseases, particularly arterial hypertension and congestive heart failure.

In one aspect of the present invention use is made of an angiotensin II type 1 receptor antagonist of the general formula I: 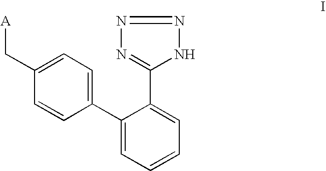
wherein A is selected from the group consisting of 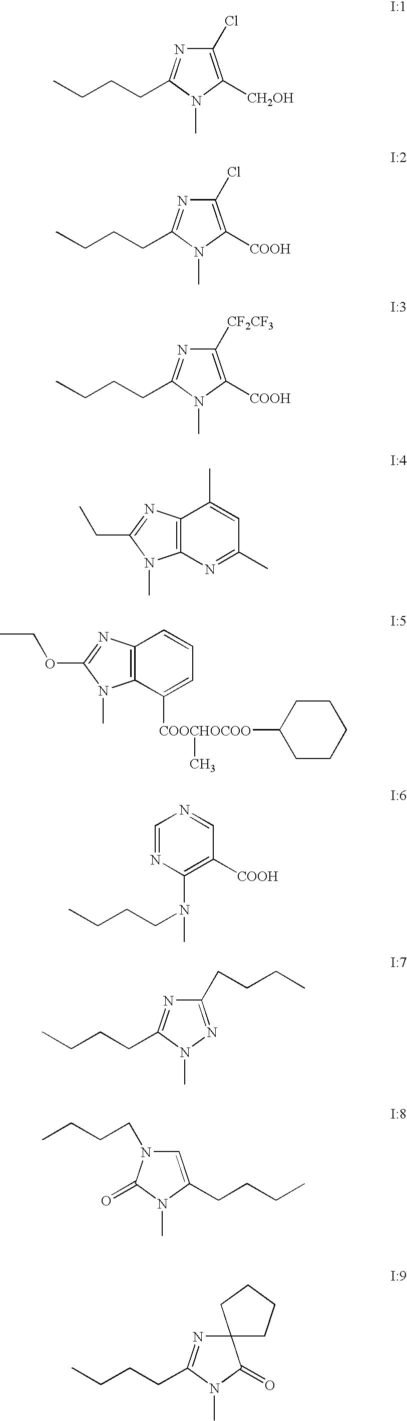 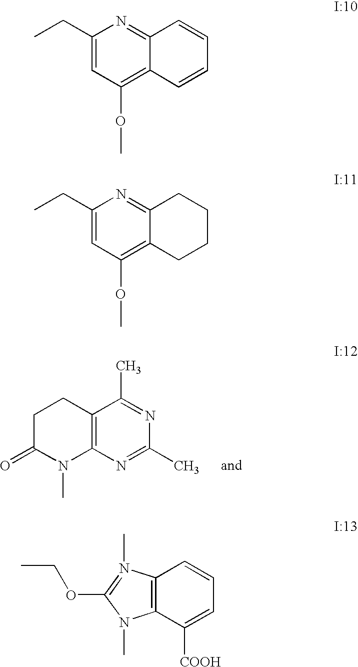
or pharmaceutically acceptable salts, solvates or stereochemical isomers of any of these, or solvates of such salts.

The compound of the general formula I wherein A is the I:1 moiety has the generic name losartan and is known from European Patent No. EP 0 253 310 B1 to du Pont.

The compound of the general formula I wherein A is the I:5 moiety has the generic name candesartan cilexetil and is known from European Patent No. 459 136 B1 and U.S. Pat. No. 5,196,444 to Takeda Chemical Industries.

The compound of the general formula I wherein A is the I:9 moiety has the generic name irbesartan.

The compound of the general formula I wherein A is the I:13 moiety has the generic name candesartan and is known from European Patent No. 459 136 B1 and U.S. Pat. No. 5,703,110 of Takeda Chemical Industries.

Further examples of angiotensin II type 1 receptor antagonists are valsartan, olmesartan, telmisartan and eprosartan.

In one aspect of the present invention, use is made of a compound of the general formula I wherein A is I:5 (candesartan cilexetil) or A is I:13 (candesartan). Candesartan cilexetil is currently manufactured and sold worldwide e.g. under the trade names Atacand®, Amias® and Blopress®.

When the angiotensin II type 1 receptor antagonists used in the present invention have several asymmetric carbon atoms, they can exist in several stereochemical forms. The present invention includes the mixture of isomers as well as the individual stereoisomers. The present invention further includes geometrical isomers, rotational isomers, enantiomers, racemates and diastereomers.

Where applicable, the angiotensin II type 1 receptor antagonists may be used in neutral form, e.g. as a carboxylic acid, or in the form of a salt, preferably a pharmaceutically acceptable salt such as the sodium, potassium, ammonium, calcium or magnesium salt of the compound at issue. Where applicable the compounds listed above can be used in hydrolyzable ester form.

Normally, the angiotensin II type 1 receptor antagonists are administered by the oral or parenteral route, e.g. by intravenous, subcutaneous or intramuscular administration. Other possible routes of administration include rectal and transdermal administration. The formulation may be given in dosage unit form, especially as tablets or capsules.

The adjuvants, diluents and carriers used in the pharmaceutical formulations of the present invention, may be conventional ones well known to the person skilled in the art. Examples of such adjuvants, diluents and carriers include substances used as binders, lubricants, fillers, disintegrants, pH regulants and thickeners as well as substances included for providing isotonic solutions.

The wording “daily dose” is defined so that the angiotensin II type 1 receptor antagonist may be given either as a unit dosage once daily, such as a tablet or a capsule, or alternatively the angiotensin II type 1 receptor antagonist may be given twice daily. The daily dose may vary within the dosage ranges mentioned below, and depends on the patient's individual response to treatment.

With the wording “therapeutic treatment” as herein used, is meant that the metabolic syndrome is treated by administering an angiotensin II type 1 receptor antagonist according to the formula I above. This means that the use of an angiotensin II type 1 receptor antagonist according to the formula I above, provides therapy of a fully or partly developed metabolic syndrome.

With the wording “prophylactic treatment” as herein used, is meant that an angiotensin II type 1 receptor antagonist according to the formula I above, may be administered to a person to prevent the metabolic syndrome.

The dose of the angiotensin II type 1 receptor antagonist and in particular a compound according to formula I to be administered in prophylaxis and/or treatment of metabolic syndrome in subjects suffering from, or susceptible to, such conditions, will depend primarily upon the angiotensin II type 1 receptor antagonists used, the route of administration, the severity of the condition to be treated and the status of the subject at issue. The daily dose, especially at oral, rectal as well as parenteral administration, can be in the range of from about 0.01 mg to about 1000 mg per day of active substance, such as from 0.1 mg to 750 mg per day of active substance or from 1 mg to 500 mg per day of active substance. In one embodiment, where candesartan and derivatives thereof are used, including candesartan cilexetil, the dosage range at oral, rectal as well as parenteral administration can be in the range of from about 0.1 mg to about 300 mg per day, such as from 0.2 mg to 200 mg or from 4 mg to 160 mg per day calculated as candesartan.

Metabolically Neutral Antihypertensive Substances

Metabolically neutral antihypertensive substances are compounds capable of reducing hypertension without influencing metabolic profile of the subject being treated. One example from this group is calcium receptor antagonists. Calcium receptor antagonists influence the inflow of calcium ions into cells, in particular into the cells of smooth muscles. The calcium antagonists are essentially dihydropyridines or or non-dihydropyridines, such as diltiazem-type or verapamil-type compounds. Examples of dihydropyridine calcium antagonists are amlodipine, verapamil, nifedipine, nimodipine, diltiazem, nicardipine, felodipine, emlodipine, ryosidine, lacidipine, niguldipine, niludipine, nisoldipine, nitrendipine, nivaldipine and isradipine, as well as, in each case, a pharmaceutically acceptable salt thereof. Examples of non-dihydropyridine calcium antagonists are flunarizine, diltiazem, mibefradil, prenylamine, fendiline, gallopamil, verapamil, tiapamil and anipamil, as well as, in each case, a pharmaceutically acceptable salt thereof.

The dose of the metabolically neutral antihypertensive, such as of a calcium receptor antagonist to be administered in prophylaxis and/or treatment of metabolic syndrome in subjects suffering from, or susceptible to, such conditions, will depend primarily upon the metabolically neutral antihypertensive used, the route of administration, the severity of the condition to be treated and the status of the subject at issue. The daily dose, e.g. at oral, rectal or parenteral administration, can be in the range of from about 1 mg to about 1000 mg per day of active substance, such as from 5 mg to 200 mg per day of active substance.

### EXAMPLES

A clinical study was performed. It was a study with a double-blind, randomised, controlled, parallel group design.

For inclusion, sitting blood pressure should be in the range of 140-179 and/or 90-104 mm Hg (mean of two measurements according to standardised procedures [1999 World Health Organization (WHO)—International Society of Hypertension Guidelines for the Management of Hypertension. Journal of Hypertension 1999; 17; 151-83] and at two visits) on placebo treatment and after the patients had been subjected to non-pharmacological treatment, as recommended [The Swedish Council on Technology Assessment in Health Care (SBU). Moderately elevated blood pressure. J Intern Med 1995; 238(Suppl 737): S1-S128; 1999 World Health Organization (WHO)—International Society of Hypertension Guidelines for the Management of Hypertension. Journal of Hypertension 1999; 17; 151-83] for one month or longer. Since the non-pharmacological intervention had been introduced before the start of the study and then maintained, its effect on metabolic variables during the study was minimal.

Exclusion criteria included: compelling indication for any particular antihypertensive drug, contraindication for any antihypertensive drug, need of lipid lowering drug therapy, severe concomitant disease, diabetes mellitus, substance abuse, or any other condition associated with poor compliance.

After four weeks of single-blind treatment with placebo, the patients were randomised to double-blind treatment with either candesartan cilexetil 16 mg or hydrochlorothiazide 25 mg and followed for one year. If sitting systolic or diastolic blood pressure was above target pressure (<130/<85 mmHg, for patients below 65 years, or <140/<90 mmHg, if 65 years or older) [1999 World Health Organization (WHO)—International Society of Hypertension Guidelines for the Management of Hypertension. Journal of Hypertension 1999; 17; 151-83] at any visit during the treatment period, double-blind treatment with felodipine extended-release 2.5-5.0 mg was added to the candesartan group and atenolol 50-100 mg was added to the hydrochlorothiazide group. No further antihypertensive treatment was allowed. Two patients were withdrawn from the study, both in the candesartan cilexetil group, since their blood pressure exceeded the pre-specified safety level (≧180 and/or ≧105 mm Hg, mean of two recordings at different visits).

To ensure that 324 patients would complete the study, it was estimated that 400 patients needed to be randomised. In all, 393 patients were randomised, 197 to candesartan cilexetil and 196 to hydrochlorothiazide; one patient was excluded due to lack of outcome data and was therefore not included in the intention-to-treat analyses. The discontinuation rates were low, 8.2 and 7.1%, respectively. No patient was lost to follow-up. In all, 370 patients out of 392 (94.4%) had never been treated with antihypertensive drugs and were thus truly newly detected hypertensives. The other 22 patients had not been drug-treated for hypertension six months before the study but short treatment periods in their past could not be excluded. Three patients (0.8%), two in the candesartan cilexetil group and one in the hydrochlorothiazide group, received lipid-lowering therapy during part of the study period and were thus protocol violators. These patients were included in the intention-to-treat but not in the per-protocol analyses.

Glucose Analyses in All Patients

Analyses of plasma glucose were carried out at the Department of Clinical Chemistry, Umeå University Hospital. Plasma glucose was routinely analysed by Vitros 950 glucose oxidase method (Ortho Clinical Diagnostics).

Lipid Analyses in All Patients

Total plasma cholesterol, LDL-cholesterol, HDL-cholesterol and triglycerides were determined in all patients at randomisation (n=392), and in most patients after six months (n=354), and after 12 months (n=352). Total plasma cholesterol and triglycerides concentrations were determined enzymatically. HDL-cholesterol was measured after the precipitation of apolipoprotein B-containing lipoproteins in whole plasma by heparin-manganese chloride [Lipid Research Clinics Program: Manual of Laboratory Operations, Bethesda, Md.: National Institutes of Health, Vol 1. Lipid and Lipoprotein Analysis. DHEW publ, 1974]. VLDL-cholesterol was assumed to equal one fifth of the plasma triglyceride concentration and LDL-cholesterol level was determined by difference according to the method of Friedewald et al [Friedewald W T, Levy R I, Fredrickson D S. Estimation of the concentration of low-density lipoprotein cholesterol in plasma, without use of the preparative ultracentrifuge. Clin Chem 1972; 18:499-502]; four patients had triglyceride values above 4.8 mmol/L (400 mg/dL). Their LDL cholesterol levels were not calculated.

The Metabolic Syndrome

The metabolic syndrome was diagnosed according to the following criteria [World Health Organization (WHO). Department of Noncommunicable Disease Surveillance. Geneva: WHO 1999 pp 1-59]:
1. Fasting plasma glucose above 6.1 mmol/L
2. Blood pressure above 140/90 mm Hg
3. One or more of the following:
   a. plasma triglycerides above 1.7 mmol/L and/or HDL below 0.9 mmol/L (men), below 1.0 mmol/L (women)
   b. Body mass index above 30 kg/m²
      Statistical Analyses

Efficacy variables were analysed using analysis of covariance (ANCOVA) with treatment and health centre as factors and baseline value as covariate. Difference in treatment effect was estimated with 95% confidence interval. To test difference between treatments in change of biochemistry variables the Wilcoxon Rank Sum test was used. With 324 patients completing the study it had an 80% power of detecting a difference in change from baseline to 12 months of 0.25 mmol/L in plasma LDL cholesterol between the groups, based on a significance level of 5% and an estimated standard deviation of the difference in change of 0.8 mmol/L. All efficacy variables were analysed according to the intention-to-treat approach. In this approach all randomised patients who had completed the study and had taken at least one dose of study drug were included.

Results

Drug Usage and Blood Pressure

In the candesartan cilexetil group, 29% were on monotherapy at the end of study whereas 71% needed add-on treatment with felodipine (mean dosage 3 mg). The corresponding figures in the hydrochlorothiazide group were 16% and 84% (atenolol, mean dosage 68 mg), respectively. Both treatment regimens lowered the blood pressure well (see table 1 below). After one year, 65% in the candesartan cilexetil group and 62% in the hydrochlorothiazide group attained a blood pressure <140/<90 mm Hg. Blood pressure at the start of the non-pharmacological treatment period, one month or more before randomisation, was 158/98 mm Hg, i.e. approximately 3/1 mm Hg higher than the randomisation pressures.

TABLE 1
Change of blood pressure and heart rate at 6 and 12 months with 95% confidence
interval of estimate and test of difference in change between treatments.

| | Candesartan cilexetil | HCTZ | 95% confidence | |
|---|---|---|---|---|
| | (n = 196) | (n = 196) | interval | P-value |
| At 6 months, sitting | | | | |
| SBP (mm Hg), mean change | −20.9 (13.1) | −23.9 (13.0) | +0.5 to +5.0 | 0.02 |
| DBP (mm Hg), mean change | −12.8 (6.9) | −13.9 (7.1) | −0.2 to +2.4 | 0.09 |
| Heart rate (bpm), mean change | −2.1 (8.4) | −6.8 (10.1) | +3.0 to +6.4 | <0.001 |
| At 12 months, sitting | | | | |
| SBP (mm Hg), mean change | −21.0 (15.2) | −22.8 (14.9) | −1.2 to +4.1 | >0.20 |
| DBP (mm Hg), mean change | −13.0 (7.4) | −12.9 (7.7) | −1.6 to +1.2 | >0.20 |
| Heart rate (bpm) mean change | −2.2 (8.4) | −7.3 (9.4) | +3.5 to +6.7 | <0.001 |

*Data are mean (SD).
Bpm = beats per minute,
DBP = diastolic blood pressure,
SBP = systolic blood pressure.

Serum Insulin, Plasma Glucose and Oral Glucose Tolerance Test

Fasting levels of both serum insulin and plasma glucose increased during treatment in the hydrochlorothiazide group in contrast to unaffected levels in the candesartan cilexetil group (see table 2 below).

TABLE 2
Insulin and glucose at baseline and 12 months with 95% confidence interval of estimate
and test of difference in change between treatments. Data are mean (SD)

| | Candesartan cilexetil | HCTZ | 95% confidence | |
|---|---|---|---|---|
| | (n = 196) | (n = 196) | interval | P-value |
| S-insulin (mlU/L) | | | | |
| Baseline | 9.25 (7.90) | 9.65 (6.09) | | |
| At 12 months | 8.96 (5.42) | 11.00 (6.88) | | |
| Mean change at 12 months | −0.30 (6.50) | 1.35 (6.09) | −2.91 to −0.61 | 0.003 |
| P-glucose (mmol/L) | | | | |
| Baseline | 5.17 (0.58) | 5.29 (0.98) | | |
| At 12 months | 5.10 (0.57) | 5.42 (0.89) | | |
| Mean change at 12 months | −0.06 (0.46) | 0.13 (0.69) | −0.34 to −0.12 | <0.001 |
| S-insulin/P-glucose | | | | |
| Baseline | 1.77 (1.38) | 1.83 (1.10) | | |
| At 12 months | 1.76 (1.06) | 2.03 (1.23) | | |
| Mean change at 12 months | −0.00 (1.23) | 0.20 (1.13) | −0.44 to 0.00 | 0.05 |

Metabolic Syndrome

At 12 months, 18 patients in the hydrochlorothiazide group vs. only five in the candesartan cilexetil group suffered from the ‘metabolic syndrome’, as defined by WHO (p=0.007); at base-line the corresponding figures were 12 and 13, respectively. The changes in fasting plasma glucose and plasma triglycerides are shown in table 3 below.

TABLE 3
Fasting plasma glucose levels and plasma triglycerides at
baseline and after 12 months for patients in the candesartan
cilexetil group suffering from the metabolic syndrome at
baseline but not after 12 months of treatment.

| Patient | Fasting glucose (mmol/L) | | Triglycerides (mmol/L) | |
|---|---|---|---|---|
| number | Baseline | 12 months | Baseline | 12 months |
| 1 | 6.4 | 5.9 | | |
| 2 | 6.1 | 5.5 | | |
| 3 | 6.5 | 5.7 | 2.15 | 1.59 |
| 4 | | | 2.34 | 1.44 |
| 5 | 6.2 | 5.7 | | |
| 6 | 6.2 | 5.6 | 2.65 | 1.28 |
| 7 | 6.2 | 5.2 | | |
| 8 | 6.2 | 6.0 | | |

1-10. (canceled)

11. A method for the treatment and/or prevention of metabolic syndrome, whereby a pharmaceutically and pharmacologically effective amount of an angiotensin II type 1 receptor antagonist alone or in combination with a metabolically neutral antihypertensive substance is administered to a subject in need of such treatment or prevention.

12. The method of claim 11, wherein the angiotensin II type 1 receptor antagonist is of the general formula I: 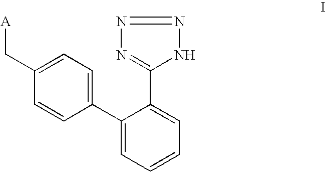
wherein A is 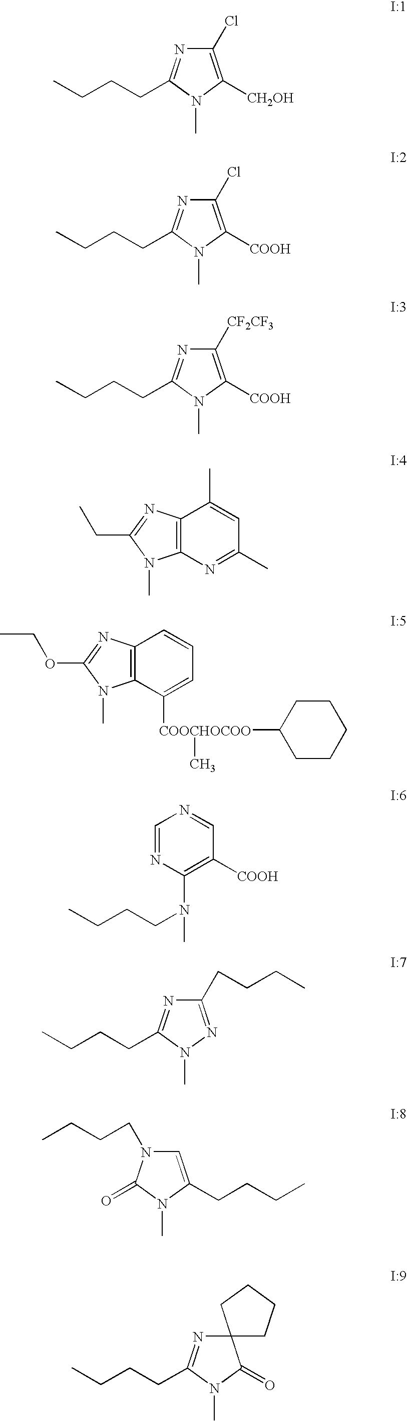 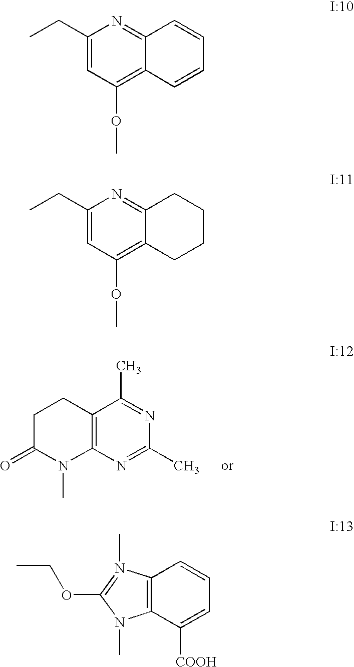
or pharmaceutically acceptable salts, solvates or stereochemical isomers thereof of any of these, or solvates of such salts.

13. The method of claim 12, wherein A is I:5.

14. The method of claim 12, wherein A is I:13.

15. The method of any one of claims 11-14, wherein the metabolically neutral antihypertensive substance is a calcium antagonist.

16. The method of claim 15, wherein the metabolically neutral antihypertensive substance is selected from amlodipine, verapamil, nifedipine, nimodipine, diltiazem, nicardipine, felodipine, emlodipine, ryosidine, lacidipine, niguldipine, niludipine, nisoldipine, nitrendipine, nivaldipine, isradipine, flunarizine, diltiazem, mibefradil, prenylamine, fendiline, gallopamil, verapamil, tiapamil and anipamil, as well as, in each case, or a pharmaceutically acceptable salt thereof.

17. The method of any one of claims 11-14, wherein the daily dose of the angiotensin II type 1 receptor antagonist is from about 0.01 mg to about 1000 mg.

18. The method of claim 17, wherein the daily dose of the angiotensin II type 1 receptor antagonist is from about 0.1 mg to 750 mg.

19. The method of claim 18, wherein the daily dose of the angiotensin II type 1 receptor antagonist is from about 1 mg to 500 mg.

20. The method of claim 13, wherein the daily dose of the angiotensin II type 1 receptor antagonist is from about 0.1 mg to about 300 mg per day calculated as candesartan.
